(19) United States
(12) Patent Application Publication
(10) Pub. No.: US 2007/0213275 A1
(43) Pub. Date: Sep. 13, 2007

(54) Formulations for ecallantide

(75) Inventors: Eliana Clark, Boylston, MA (US); Thomas Beck, Concord, MA (US); John T. Ghiorse III, Douglas, MA (US); Jane Mansfield, Wareham, MA (US); Elizabeth Devan, Shirley, MA (US)

Correspondence Address: FISH & RICHARDSON PC, P.O. BOX 1022, MINNEAPOLIS, MN, 55440-1022, US

(73) Assignee: DYAX CORP.

(21) Appl. No.: 11/716,278

(22) Filed: Mar. 9, 2007

Related U.S. Application Data
(60) Provisional application No. 60/781,444, filed on Mar. 10, 2006.

(51) Int. Cl. A61K 38/17; A61K 31/7012

(52) U.S. Cl. 514/12; 514/53

(57) ABSTRACT

Disclosed herein are new formulations for ecallantide which are stable at room temperature and useful as pharmaceutical formulations.

### CROSS-REFERENCE TO RELATED APPLICATIONS

Under 35 U.S.C. § 119(e)(1), this application claims priority to Provisional Patent Application No. 60/781,444, entitled “FORMULATIONS FOR ECALLANTIDE,” filed on Mar. 10, 2006, the entire contents of which are hereby incorporated by reference.

### BACKGROUND

Ecallantide is a 60 amino acid peptide which has the general structure of a Kunitz domain. Ecallantide has been shown to be a potent inhibitor of plasma kallikrein.

### SUMMARY OF THE INVENTION

Disclosed herein are new formulations for ecallantide which are stable at room temperature and useful as pharmaceutical formulations.

The disclosure provides compositions containing ecallantide (“ecallantide formulations”), including a buffering agent, a buffering agent/cryoprotectant, and ecallantide. The buffering agent may be a histidine or phosphate buffer which buffers the pH to between about 6.0 and 7.0, and the bulking agent may be sucrose or a combination of sucrose and mannitol. In some instances the bulking agent/cryoprotectant also includes dextran, such as dextran 40.

Further provided are compositions made by the methods disclosed herein.

In some embodiments, the buffering agent is histidine, which may be present at 10 mM. In some embodiments, the formulation has a pH of about 6.5.

In some embodiments, the bulking agent/cryoprotectant is sucrose, which may be present at 10% (w/v).

In some embodiments, the ecallantide is present at 10 mg/mL, 20 mg/mL, or 30 mg/mL.

In some embodiments, the formulations are isotonic.

The ecallantide formulations disclosed herein may be lyophilized. Accordingly, the disclosure provides lyophilized formulations for ecallantide including a buffering agent, a buffering agent/cryoprotectant, and ecallantide. The buffering agent may be a histidine or phosphate buffer which buffers the pH to between about 6.0 and 7.0, and the bulking agent may be sucrose or a combination of sucrose and mannitol. In some instances the bulking agent/cryoprotectant also includes dextran, such as dextran 40.

The components of lyophilized ecallantide formulations may be present at varying molar ratios, such as about 1:1 to about 7.5:1 or about 2:1 to about 2.5:1 (buffering agent:ecallantide), or about 250:1 to about 45:1 or about 75:1 to about 60:1 (bulking agent/cryoprotectant:ecallantide), or about 2.5:75:1 to about 2:65:1, or about 7:208:1, about 2.4:70:1, or about 1.4:41:1 (buffering agent:bulking agent/cryoprotectant:ecallantide).

The components of lyophilized ecallantide formulations may be present at varying percentages (w/w), such as about 1% to about 2% (w/w) buffering agent, about 90% to about 60% bulking agent/cryoprotectant, and about 9% to about 37% ecallantide.

Also provided herein are methods for making the lyophilized ecallantide formulations disclosed herein, by obtaining or producing a mixture of a buffering agent, a bulking agent/cryoprotectant, and ecallantide, and lyophilizing the mixture.

Also provided are methods for treating angioedema (hereditary angioedema, angiotensin converting enzyme (ACE) inhibitor-induced angioedema, acquired (e.g., C1 esterase inhibitor deficiency) angioedema, idiopathic chronic angioedema, allergic angioedema, and nonsteroidal anti-inflammatory drug (NSAID) induced angioedema) by administering an effective amount of an ecallantide formulation of the disclosure to a subject having or suspected of having angioedema.

Also provided are kits including the ecallantide formulations of the disclosure. The kits include at least one container inducing an ecallantide formulation of the disclosure, and may also include instructions regarding the use of the ecallantide for the treatment of angioedema. The container may be an ampoule, vial, prefilled syringe, or an autoinjection device (or cartridge for an autoinjection device).

### BRIEF DESCRIPTION OF THE DRAWINGS

FIG. 1 shows a graphical depiction of plasmid pPIC K503.

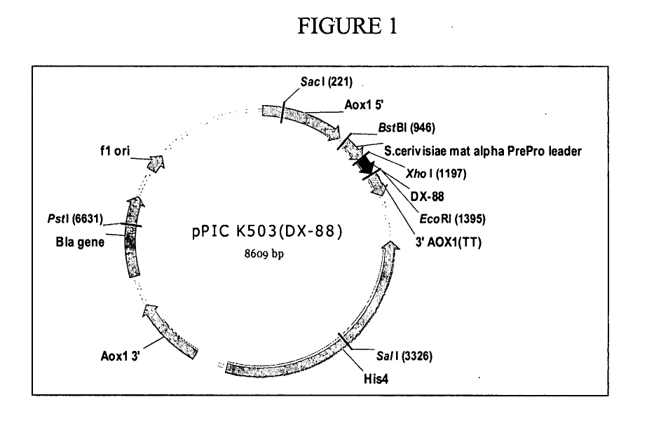

FIG. 2 shows a graphical depiction of RP-HPLC data measuring pyroglutamate levels in formulations buffered with PBS (panel A) or 10 mM histidine (panel B).

FIG. 3 shows a graphical depiction of RP-HPLC data measuring pyroglutamate levels (panel A) and peak 4 levels (panel B).

### DETAILED DISCLOSURE OF THE INVENTION

Disclosed herein are new formulations for ecallantide which are stable at room temperature and useful as pharmaceutical formulations.

As used herein, the word “about,” when used in relation to a percentage, a molar concentration, or a molar ratio, indicates a range of plus or minus 10% surrounding the indicated value (e.g., ‘about 10 mM’ means 9 mM to 11 mM). When “about” is used in relation to a pH value, it indicates a range of plus or minus 0.2 pH units surrounding the indicated value (e.g., ‘about pH 7.0’ means pH 6.8 to 7.2).

Ecallantide

A number of Kunitz domain-based proteins are known in the art, for example: U.S. Pat. Nos. 4,245,051; 5,278,285; 5,436,153; 5,728,674; 5,563,123; 5,589,359; 5,696,088, 5,663,143; 5,880,256; 5,968,897; 5,977,057; 6,103,500; 5,990,079; 6,063,764; 6,414,124; 6,583,108; 6,593,291; and 6,914,135.

Ecallantide is a 60 amino acid peptide which has the general structure of a Kunitz domain. Ecallantide has the sequence Glu Ala Met H is Ser Phe Cys Ala Phe Lys Ala Asp Asp Gly Pro Cys Arg Ala Ala H is Pro Arg Trp Phe Phe Asn Ile Phe Thr Arg Gln Cys Glu Glu Phe Ile Tyr Gly Gly Cys Glu Gly Asn Gln Asn Arg Phe Glu Ser Leu Glu Glu Cys Lys Lys Met Cys Thr Arg Asp (SEQ ID NO:1). The molecular weight of ecallantide is 7,054 Daltons. Ecallantide is a highly effective inhibitor of plasma kallikrein, and has been proposed as a therapeutic for a number of indications, including hereditary angioedema and prevention of ischemia (Williams et al., 2003, Transfus. Apher. Sci. 29(3):255-58; U.S. 2004/0038893).

Ecallantide may be made synthetically using any standard polypeptide synthesis protocol and equipment. For example, the stepwise synthesis of ecallantide may be carried out by the removal of an amino (N) terminal-protecting group from an initial (i.e., carboxy-terminal) amino acid, and coupling thereto of the carboxyl end of the next amino acid in the sequence of the polypeptide. This amino acid is also suitably protected. The carboxyl group of the incoming amino acid can be activated to react with the N-terminus of the bound amino acid by formation into a reactive group such as formation into a carbodiimide, a symmetric acid anhydride, or an “active ester” group such as hydroxybenzotriazole or pentafluorophenyl esters. Useful solid-phase peptide synthesis methods include the BOC method, which utilizes tert-butyloxycarbonyl as the a-amino protecting group, and the FMOC method, which utilizes 9-fluorenylmethloxycarbonyl to protect the a-amino of the amino acid residues. Both methods are well known to those of skill in the art (Stewart, J. and Young, J., Solid-Phase Peptide Synthesis (W.H. Freeman Co., San Francisco 1989); Merrifield, J., 1963. Am. Chem. Soc., 85:2149-2154; Bodanszky, M. and Bodanszky, A., The Practice of Peptide Synthesis (Springer-Verlag, New York 1984), the entire teachings of these references is incorporated herein by reference).

Alternatively, ecallantide may be produced by recombinant methods using any of a number of cells and corresponding expression vectors, including but not limited to bacterial expression vectors, yeast expression vectors, baculovirus expression vectors, mammalian viral expression vectors, and the like. Ecallantide may also be produced transgenically using nucleic acid molecules comprising a sequence encoding ecallantide, wherein the nucleic acid molecule can be integrated into and expressed from the genome of a host animal using transgenic methods available in the art. In some cases, it may be necessary or advantageous to fuse the coding sequence for ecallantide to another coding sequence in an expression vector to form a fusion polypeptide that is readily expressed in a host cell. Preferably, the host cell that expresses such a fusion polypeptide also processes the fusion polypeptide to yield only the desired amino acid sequence (i.e., ecallantide). Obviously, if any other amino acid(s) remain attached to the expressed ecallantide, such additional amino acid(s) should not diminish the activity of the ecallantide so as to preclude use of the polypeptide in the formulations disclosed herein.

A particular method of producing ecallantide disclosed in the Examples utilizes recombinant expression in yeast host cells. A yeast expression vector, which permits a nucleic acid sequence encoding the amino acid sequence of ecallantide to be linked in the same reading frame with a nucleotide sequence encoding the matα prepro leader peptide sequence of Saccharomyces cerevisiae, which in turn is under the control of an operable yeast promoter. The resulting recombinant yeast expression plasmid is then transformed by standard methods into the cells of an appropriate, compatible yeast host, which cells are able to express the recombinant protein from the recombinant yeast expression vector. Preferably, a host yeast cell transformed with such a recombinant expression vector is also able to process the fusion protein to provide active ecallantide useful in the methods and compositions disclosed herein. Yeast host cell useful for producing recombinant ecallantide in such methods is Pichia pastoris.

Ecallantide for use in pharmaceutical formulations should be substantially homogenous. Accordingly, ecallantide is normally purified following production (by synthesis or recombinant expression). Ecallantide purification may be carried out using techniques known in the art, including size-exclusion chromatography, ion exchange (anion and/or cation exchange) chromatography, hydrophobic interaction chromatography, affinity chromatography, and reverse-phase chromatography, or any combination thereof. Additionally, buffer exchange and/or concentration technologies may be used, when desired.

As described in the Examples herein, ecallantide is unstable under certain conditions, giving rise to both high molecular weight (e.g., aggregation products) and low molecular weight (e.g., fragmentation products) degradation products, as well as modification products (e.g., amino-terminal pyroglutamate), upon storage. The formulations disclosed herein substantially stabilize ecallantide, preventing or reducing formation of aggregation products, fragmentation products, or modification products.

Ecallantide may be present in the instant formulations at varying levels, depending on the intended use (e.g., the intended dose). In liquid formulations, ecallantide may be present at concentrations ranging from about 5 mg/mL (0.7 mM) to about 50 mg/mL (7 mM), or about 7 mg/mL (1 mM) to about 40 mg/mL (5.7 mM), or about 10 mg/mL (1.4 mM) to about 30 mg/mL (4.2 mM), or about 30 mg/mL. Expressed as percentage (w/v), ecallantide may be present at concentrations ranging from about 0.5% to about 5%, or about 0.7% to about 4%, or about 1% to about 3%. In lyophilized formulations, ecallantide may be present at about 5% to about 45% (w/w), or about 7% to about 40% (w/w) or about 9% to about 37% (w/w).

pH and Buffering Agent

The formulations disclosed herein are pH controlled with a buffering agent. As described in the Examples, ecallantide is stable in the pH range of about 6.0 to about 7.0. Accordingly, provided herein are formulations which, when in liquid form (e.g., when produced or when reconstituted), have a pH of about 6.0 to about 7.0, for example about 6.0 (e.g., pH 5.8 to 6.2), about 6.5 (e.g., pH 6.3 to 6.7), or about 7.0 (e.g., pH 6.8 to 7.2).

Any buffering agent that is suitable for buffering in the range of pH about 6.0 to about 7.0 may be used. In some embodiments, the buffer is also pharmaceutically acceptable. Suitable buffers include citrate, succinate, malate, cacodylate, 2-(N-morpholino)ethanesulfonic acid hydrate (MES), citrate, maleate, histidine, phosphate, and carbonate. In certain embodiments, the buffering agent is histidine or phosphate. In certain embodiments the buffering agent is histidine.

The buffering agent is included at a concentration which provides sufficient pH control under the expected conditions of storage and (for lyophilized formulations) reconstitution. For formulations in liquid form, the buffering agent is generally included at about 3 mM to about 20 mM, or about 5 mM to about 15 mM, or about 8 mM to about 12 mM, or about 10 mM. When calculated as a percentage (w/v), the buffering agent may be present at concentration of about 0.045% to about 0.31%, or about 0.08% to about 0.23%, or about 0.12% to about 0.19%. or about 0.15%. For lyophilized formulations, the buffering agent is generally included at about 0.25% to about 5% (w/w), or about 0.5% to about 2.5% (w/w), or about 1% to about 2% (w/w).

Bulking Agent/Cryoprotectant

The formulations disclosed herein include a bulking agent/cryoprotectant. The inventors have discovered that sucrose, alone or combined with mannitol, is useful as a bulking agent/cryoprotectant for ecallantide formulations. Additionally, the formulations may include dextran, which in some embodiments is dextran 40.

Unexpectedly, the inventors have also found that trehalose, a commonly used bulking agent/cryoprotectant that would be expected to be stabilizing, is destabilizing when included in ecallantide formulations. Accordingly, the formulations disclosed herein may be substantially or entirely free of trehalose, as the inventors have discovered that trehalose destabilizes ecallantide formulations. As used herein, “substantially free of trehalose” means that the formulation (in liquid form) is less than 1 mM in trehalose or (in lyophilized form) less than 1% trehalose by weight.

Bulking agent/cryoprotectant is included in the instant formulations in an amount that provides sufficient bulk when dried to produce an acceptable lyophilized cake and to provide at least a measure of cryoprotection to the ecallantide. In liquid formulations, when measured as a percentage of the formulation, the bulking agent/cryoprotectant is present at about 3% to about 15% (w/v), or about 4% to about 15%, or about 5% to about 10%. In liquid formulations, when measured as molarity of the bulking agent/cryoprotectant, the bulking agent/cryoprotectant is present at about 200 mM to about 350 mM, or about 250 mM to about 300 mM. In embodiments in which the bulking agent/cryoprotectant is sucrose, the bulking agent/cryoprotectant may be present at about 292 mM. In lyophilized formulations, the bulking agent is present at about 95% to about 55% (w/w), or about 90% to about 60% (w/w).

Formulations

The formulations disclosed herein comprise ecallantide, a pH buffering agent and a bulking agent/cryoprotectant. Because an intended use of the formulations is as pharmaceutical formulations, in certain embodiments, the formulations are isotonic (e.g., have an osmolarity of between 250 to 350 mOsM, or about 300 mOsM). As will be understood by those in the art, the ratios of the components will vary according to the concentration of the components, particularly the ecallantide (which may be varied according to the intended dosage). For pharmaceutical applications, the components of the formulations disclosed herein should be U.S. Pharmacopeia (USP) or like grade, or produced in accordance with Good Manufacturing Practices (GMP).

In liquid form, the amounts of the components of the formulations are can be easily described by molar or percentage (w/v) concentrations. When expressed in molar concentrations, the instant formulations may be about 3 mM to about 20 mM, or about 5 mM to about 15 mM, or about 8 mM to about 12 mM, or about 10 mM in buffering agent, about 200 mM to about 350 mM, or about 250 mM to about 300 mM, or about 292 mM in bulking agent/cryoprotectant, and about 1 mM to about 5 mM, or about 1.4, 2.8, or 4.2 mM in ecallantide. When expressed as percentage (w/v) concentrations, the formulations may be 0.045% to about 0.31%, or about 0.08% to about 0.23%, or about 0.12% to about 0.19%. or about 0.15% in buffering agent, 3% to about 15%, or about 4% to about 15%, or about 5% to about 10% in bulking agent/cryoprotectant, and about 0.5% to about 5%, or about 0.7% to about 4%, or about 1% to about 3% ecallantide.

In dried (e.g., lyophilized) form, the amounts of the components are most easily described as percentages (w/w) or as molar ratios. When expressed as percentages, the instant formulations may be about 0.25% to about 5% (w/w), or about 0.5% to about 2.5% (w/w), or about 1% to about 2% (w/w) in buffering agent, about 95% to about 55% (w/w), or about 90% to about 60% (w/w) in bulking agent/cryoprotectant, and 5% to about 45% (w/w), or about 7% to about 40% (w/w) or about 9% to about 37% (w/w) in ecallantide. As will be understood by those of ordinary skill in the art, the sum of the percentage amount of the buffering agent, bulking agent/cryoprotectant, and the ecallantide may be, and in fact will commonly be, less than 100%, with the balance being retained solvent. When expressed as molar ratios (buffering agent:bulking agent/cryoprotectant:ecallantide), the instant formulations may be from about 7.5:208:1 to about 1:45:1, or from about 2:100:1 to about 2.5:75:1, or about 7:208:1, or about 2.4:70:1, or about 1.4:41:1.

One exemplary formulation includes (in liquid form) about 10 mM histidine as the buffering agent, about 10% (w/v) sucrose as the bulking agent/cryoprotectant, and about 10 mg/mL ecallantide and is at pH 6.5. In dried (lyophilized) form, this formulation is about 1.4% (w/w) buffering agent, 88.8% (w/w) bulking agent/cryoprotectant, and about 8.9% (w/w) ecallantide, and has a molar ratio of about 7:208:1 (histidine:sucrose:ecallantide).

Another exemplary formulation includes (in liquid form) about 10 mM histidine as the buffering agent, about 10% (w/v) sucrose as the bulking agent/cryoprotectant, and about 30 mg/mL ecallantide, and is at pH 6.5. In dried (lyophilized) form, this formulation is about 1.2% buffering agent, about 75.4% bulking agent/cryoprotectant, and about 22.6% ecallantide, and has a molar ratio of about 2.4:70:1.

The formulations disclosed herein may be manufactured by conventional techniques which yield the desired final composition. The components may be dissolved directly in water to their final concentrations, or may be made up as concentrates which are combined and diluted to generate the final composition. Alternately, buffer exchange techniques may be used.

Commonly, the ecallantide will be in an aqueous solution, as a consequence of the final processing step of the ecallantide production. This ecallantide solution may then be buffer exchanged (e.g., by diafiltration) to yield the desired formulation or, when buffer exchange is not feasible (e.g., when the bulking agent/cryoprotectant renders the formulation too viscous for buffer exchange), the ecallantide may be buffer exchanged (and concentrated if necessary) to render a concentrated solution which is then mixed with the remaining components to produce the desired formulation (e.g., for a desired formulation that is 10 mM histidine, pH 6.5, 10% sucrose, and 30 mg/mL ecallantide, the ecallantide solution is buffer exchanged and concentrated as necessary to make a stock which, when mixed with a concentrated sucrose solution or even dry sucrose, yields the final formulation of 10 mM histidine, pH 6.5, 10% sucrose, and 30 mg/mL ecallantide).

Lyophilization

Lyophilization, or freeze-drying, is a process in which a liquid composition is frozen, then dehydrated by sublimation of the frozen liquid (e.g., water). The sublimation is accomplished at a temperature suitable for primary drying. A temperature suitable for primary drying is one that maintains the product at a temperature that is below the eutectic point or the collapse temperature of the formulation.

The material to be lyophilized (e.g., the ecallantide formulation) may be frozen prior to loading into the lyophilization apparatus, or may be loaded into the apparatus in liquid form, and frozen while in the machine. Freezing of the liquid formulation may be carried out in any fashion, including a single step down to the desired temperature, as a single ramp (e.g., continuosly decreasing temperature down to the desired temperature), or in a series of steps/ramps. The ‘desired temperature’ for the frozen liquid formulation may be any temperature at which the material is frozen, but is commonly lower than the freezing point of the material, and may range from about 0° C. to about −50° C. Once the desired temperature is reached (or following an equilibration period after reaching the desired temperature), the partial vacuum is established, which may range from about 50 to about 250 mTorr, or about 60 to about 200 mTorr, or about 75 to about 100 mTorr.

The temperature within the lyophilization apparatus may be held constant during the lyophilization process, but is more commonly adjusted (generally increased) during the process. For example, a lyophilizer may be equilibrated to about −40° or about −45° C. before the vacuum is applied, then gradually warmed in a series of steps or ramps as the primary drying phase of the lyophilization process proceeds. For example, for a lyophilization process that begins at about −40° C., the lyophilizer may be stepped/ramped up through a series for sub-freezing temperatures during the initial portion of the primary drying phase (e.g., in a series of about 5° or 10° C. increments or in a series of irregular steps, such as from about −40° C. to about −35°, then to about −25° C., then to about −10° C., or from about −40° C. to about −30° C., then to about −15° C., or from −40° C. to about −30° C., then to about −25° C., or from about −40° C. to about −25°). The later stages of primary drying may be carried out at same temperature or an increased temperature, such as a temperature between about 0° C. to about 10° C. (e.g., about 3°, about 5°, about 7°, or about 10° C.).

The exact formulation, size and type of the container holding the sample (e.g., glass vial), the volume of liquid, and the lyophilization temperature and pressure will mainly dictate the time required for drying, which can range from a few hours to several days (e.g. 40-60 hrs). Exemplary primary drying conditions include (1) a vacuum level of 75 mTorr, a temperature of about −25° C. for the bulk of the primary drying stage, followed by a period at about 5° C., and a primary drying time of about 30-35 hours, and (2) a vacuum level of 75 mTorr, a temperature of about −25° C. for the primary drying stage, and a primary drying time of about 15-20 hours, and.

A secondary drying stage may be carried out, depending primarily on the type and size of container and the exact formulation employed. In some instances, a secondary drying stage at elevated temperature (e.g., about 0° C. to about 40° C., or about 10° C. to about 30° C., or about 20° or about 30° C.) will be employed. However, in some instances, a secondary drying step may not be necessary. The time and pressure required for secondary drying will be that which produces a suitable lyophilized cake. Accordingly, the secondary drying conditions (and the need for a secondary drying step at all) are dependent on the temperature and other parameters. The secondary drying time is dictated by the desired residual moisture level in the product and typically takes at least about 5 hours (e.g. about 5 to about 20 hours, such as about 8, about 9, about 10, about 12, about 15, or about 18 hours). The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

Following lyophilization (and any transfer step, if required), the lyophilized formulation is typically sealed into its container. Sealing can be with a non-resilient closure (e.g., melting the end of an all glass vial to close the vial) or by installation of a resilient closure (e.g., by closing the opening of the container with a resilient stopper, which may be then be secured by crimping of a seal holding the stopper in place). In some instances, the containers will be sealed under conditions that render the contents under reduced pressure and/or reduced oxygen tension (e.g., as would be accomplished by sealing the containers in a reduced pressure nitrogen environment).

As a general proposition, lyophilization will result in a lyophilized formulation in which the moisture content thereof is less than about 5%, for example, less than about 3%, or less than about 2%.

Reconstitution and Administration

At the desired stage, typically when it is time to administer the protein to the patient, the lyophilized formulation may be reconstituted with a diluent. The volume of diluent used for reconstitution is the volume that will yield a reconstituted formulation with the desired ecallantide concentration. In some embodiments, the lyophilized formulation is reconstituted (e.g., an appropriate amount of diluent is added) to yield a reconstituted formulation with 10, 20, 30, or 40 mg/mL ecallantide, and in certain embodiments, the lyophilized formulation is reconstituted to yield a reconstituted formulation with 30 mg/mL ecallantide with 10 mM histidine, pH 6.5, and 10% sucrose (w/v).

Exemplary diluents include sterile water for injection (WFI), and bacteriostatic water for injection (BWFI), although other diluents, such as a pH buffered solution (e.g. phosphate-buffered saline), sterile saline solution, Ringer's solution or dextrose solution may be used.

The diluent optionally contains a preservative. Useful preservatives include aromatic alcohols such as benzyl or phenol alcohol. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0%, about 0.5-1.5%, or about 1.0-1.2%.

Reconstitution of lyophilized formulations generally takes place at room temperature (e.g., 20° to 25° C.) to ensure complete hydration, although other temperatures may be employed as desired. The time required for reconstitution will depend on the exact constituents of the formulation (e.g., the type of diluent, amount of excipient(s) and ecallantide). Reconstitution may be carried out manually (e.g., by the manual addition of diluent to the lyophilized formulation by injection through an injection port into the container containing the lyophilized formulation) or automatically (e.g., by the automatic addition of the diluent to the lyophilized formulation in a device configured for automatic reconstitution, such as the Becton-Dickinson BD™ Liquid Dry Injector).

The formulations (liquid and reconstituted lyophilized) are useful as pharmaceutical formulations, generally for parenteral administration. Parenteral administration includes, but is not limited to, intravenous (IV), intramuscular (IM), subcutaneous (SC), intraperitoneal (IP), intranasal, and inhalant routes. IV, IM, SC, and IP administration may be by bolus or infusion, and in the case of SC, may also be by slow release implantable device, including, but not limited to pumps, slow release formulations, and mechanical devices. The dose, route, and method of administration will depend on the disorder to be treated and the medical history of the patient.

Methods of Use

Also provided herein are methods of treating disorders associated with excess or unregulated plasma kallikrein activity utilizing the formulations disclosed herein. As used herein, the term “treating” refers to stabilizing, ameliorating, improving, or eliminating a symptom of the disorder to be treated. A number of clinical disorders are associated with excess/dysregulated plasma kallikrein activity, including hereditary angioedema (including types I, II, and III hereditary angioedema), angiotensin converting enzyme (ACE) inhibitor-induced angioedema, acquired (e.g., C1 esterase inhibitor deficiency) angioedema, idiopathic chronic angioedema, allergic angioedema, and nonsteroidal anti-inflammatory drug (NSAID) induced angioedema (collectively, hereditary, ACE inhibitor-induced, idiopathic chronic, allergic, and NSAID-induced angioedema are referred to herein as “angioedemas”). Administration of an ecallantide formulation of the disclosure results in stabilization, amelioration, improvement, or elimination of at least one symptom (e.g., localized edema) of the angioedema being treated.

Accordingly, the disclosure provides (1) methods of treating hereditary angioedema by administering an effective amount of an ecallantide formulation disclosed herein to a subject having or suspected of having hereditary angioedema, (2) methods of treating ACE inhibitor-induced angioedema by administering an effective amount of an ecallantide formulation disclosed herein to a subject having or suspected of having ACE inhibitor-induced angioedema, (3) methods of treating acquired (e.g., C1 esterase inhibitor deficiency) angioedema by administering an effective amount of an ecallantide formulation disclosed herein to a subject having or suspected of having acquired angioedema, (4) methods of treating idiopathic chronic angioedema by administering an effective amount of an ecallantide formulation disclosed herein to a subject having or suspected of having idiopathic chronic angioedema, (5) methods of treating allergic angioedema by administering an effective amount of an ecallantide formulation disclosed herein to a subject having or suspected of having allergic angioedema, and (6) methods of treating NSAID-induced angioedema by administering an effective amount of an ecallantide formulation disclosed herein to a subject having or suspected of having NSAID-induced angioedema. In some instances, the method of treatment may further include reconstituting the lyophilized ecallantide formulation prior to administration.

The amount of an ecallantide formulation that supplies an effective amount may vary according to the medical history of the patient and the severity of the disease (or acute attack or exacerbation of the disease). In some embodiments, the effective amount of an ecallantide formulation is an amount that contains 30 mg of ecallantide.

In accordance with the instant methods, the ecallantide formulation may be administered by any parenteral route. In certain embodiments the ecallantide formulation is administed by subcutaneous bolus injection.

The ecallantide formulation may be administered to the subject by a person other than the subject (e.g., a medical professional) or it may be self-administered by the subject. Any device compatible with the selected mode of administration may be used, including syringes, infusion pumps, intravenous or subcutaneous catheters, and auto-injection devices.

Kits

Further provided are kits including the formulations disclosed herein. The kits disclosed herein include one or more packages containing a formulation of the disclosure, and may further include instructions relating to the use of the formulation (e.g., for the treatment of angioedemas). The instructions included with the kit, which are typically written, but may be electronic (and may include links to one or more sites on the world wide web) generally include information as to dosage, dosing schedule, and route of administration for the treatment of angioedemas. The packages of the ecallantide formulation may be unit doses, bulk packages (e.g., multi-dose packages) or sub-unit doses.

The ecallantide formulation packages may be in any packaging appropriate to the intended use. For liquid formulations, appropriate packages include, but are not limited to, ampoules with resilient stoppers, ampoules with non-resilient closures (e.g., sealed glass ampoules), prefilled syringes, and auto-injection devices, such as a Bioject IJECT® needless injector or DIAPEN® injector, as well as cartridges for autoinjectors. For lyophilized formulations, appropriate packages include, but are not limited to, ampoules with resilient stoppers, devices for self-administration (e.g., a BD® Liquid Dry Injector, which provides automated reconstitution and injection), and prefilled syringes.

The following examples are intended to illustrate, but not limit, the instant disclosure.

### EXAMPLES

### Example 1

### Production of Ecallantide

Ecallantide was produced by recombinant expression in yeast (P. pastoris). A sequence encoding a fusion of the signal sequence from S. cerevisiae prepro-matα and ecallantide was cloned into the AOX1 region of a plasmid derived from pHIL-D2 (which carries an ampicillin resistance gene and HIS4), to create pPIC-K503.

Spheroplasts of P. pastoris strain GS115 having the His4⁻ phenotype were transformed with the linearized (at the SacI site) pPIC-K503, followed by homologous recombination of the plasmid DNA into the host 5′ AOX1 locus. The plasmid inserted into the AOX1 locus of the host cells, converting them to a His4⁺ phenotype, and making the ecallantide expression cassette controlled by the AOX1 locus.

Recombinant strains were selected by growth in the absence of exogenous histidine with methanol as the sole carbon source. Selected colonies were cloned, and expression studies were carried out to identify clones secreting the high levels of ecallantide into the culture medium. A working cell bank was created using a high-expressing clone.

An inoculum culture was established by inoculating flasks containing sterile inoculum broth (yeast nitrogen base, potassium phosphate, and glycerol, pH=5) with cells from the working cell bank. The inoculum cultures were incubated at 30° C. for approximately 20 hours.

The inoculum culture was used to inoculate the seed fermentation culture. The seed fermentation culture was grown in a defined medium (orthophosphoric acid, calcium sulfate, potassium sulfate, magnesium sulfate, potassium hydroxide, glycerol, d-biotin, metal salts (sulphuric acid, copper sulfate, sodium iodide, manganese sulfate, sodium molybdate, boric acid, cobalt chloride, zinc chloride, and iron sulfate), an antifoam solution, and ammonium hydroxide) and was run at 30° C. to an OD₆₀₀ of 28 to 56 in a fermenter.

The seed fermentation culture was then used to inoculate a production fermentation culture. The seed fermentation culture was added to pre-warmed production fermentation medium (orthophosphoric acid, glycerol, calcium sulfate, potassium sulfate, magnesium sulfate, potassium hydroxide, metal salts (sulphuric acid, copper sulfate, sodium iodide, manganese sulfate, sodium molybdate, boric acid, cobalt chloride, zinc chloride, and iron sulfate), an antifoam solution, and ammonium hydroxide), d-biotin, an antifoam solution, and ammonium hydroxide) in a fermenter, and expanded in the glycerol batch phase until the initial glycerol in the medium was exhausted. The culture was then switched to a glycerol batch-fed phase, in which glycerol was added to the medium, to allow further expansion of the production strain. Finally, the culture was switched to the mixed feed phase, by switching to a glycerol and methanol feed, for approximately 83 hours.

All fermentation stages were carried out with agitation and aeration (with addition of oxygen if necessary).

The fermenter contents were cooled and diluted with purified water. The initial purification step utilized expanded bed chromatography (EBC) to capture the ecallantide from the diluted fermenter broth and to remove the yeast from the fermentation. The diluted fermenter culture was loaded onto an expanded bed column (STREAMLINE™ SP resin) in down flow mode, washed in up-flow mode, allowed to settle, then washed and eluted in down-flow mode.

Further purification was carried out by a series of column chromatography steps operated in bind/wash/elute format. EBC eluate was loaded onto a cation exchange (CEX) resin (Bio-Rad MACRO-PREP® High S), which washed and eluted. The CEX eluate was adjusted to be 1.1 M in ammonium sulfate, then loaded onto a hydrophobic interaction chromatography (HIC) resin, which washed and eluted. The HIC eluate was buffer exchanged by ultrafiltration/diafiltration with 1 kDa MWCO regenerated cellulose membranes (UFDF), then loaded onto an anion exchange (AEX) chromatography resin (BioSepra Q HYPERD®). which washed, then eluted. The AEX eluate was buffer exchanged into PBS, pH 7.0 by UFDF, aseptically filtered through 0.22 μm membranes, and dispensed aseptically into sterile PETG bottles and stored at −20° C.

### Example 2

### pH and Buffering Agent Selection

Ecallantide stability was examined at pH 6.0, 6.5 and 7.0 in a variety of buffers. Ecallantide (10 mg/mL in an isotonic phosphate buffered saline solution, pH 7.0) was buffer-exchanged by dialysis into (a) 10 mM succinate, pH 6.0, 150 mM NaCl, (b) 10 mM histidine, pH 6.0, 150 mM NaCl, (c) 10 mM histidine, pH 6.5, 150 mM NaCl, (d) phosphate buffered saline (PBS, 4.3 mM sodium phosphate, 1.5 mM potassium phosphate, 137 mM NaCl), pH 6.5, or (e) 10 mM histidine, pH 7.0, 150 mM NaCl.

Samples of each formulation were sterile filtered into individual tubes and stored at 4° or 30° C. for six weeks, and samples were analyzed at 1, 2, 3.5, 5, and 6 weeks by HPLC size exclusion chromatography (SEC) to detect aggregate formation and fragmentation, and reverse phase (RP) HPLC to detect pyroglutamic acid formation.

SEC-HPLC results showed increasing high molecular weight species (aggregates) with increasing pH. Conversely, low molecular weight species (fragments) decreased with increasing pH.

RP-HPLC analysis showed significantly greater pyroglutamate production in pH 7.0 samples. Among the pH 6.0 and 6.5 samples, the pH 6.5 PBS sample had slightly higher levels of pyroglutamate.

### Example 3

### Bulking Agent/Cryoprotectant Selection for Low Dose Ecallantide

Ecallantide stability was examined in lyophilized formulations utilizing different bulking agent/cryoprotectant schemes. Ecallantide (10 mg/mL in PBS, pH 7.0) was buffer-exchanged by dialysis into formulations buffered with either 10 mM histidine, pH 6.5 or PBS, pH 6.5 and including (a) 5% mannitol, (b) 3% mannitol/3% sucrose, (c) 10% sucrose, or (d) 7.5% sucrose/5% dextran 40 as a bulking agent/cryoprotectant.

Samples of each formulation were sterile filtered into glass vials, frozen, lyophilized, then stored at 4° or 40° C. for eight weeks. Samples were reconstituted with water, then assayed by SEC-HPLC and RP-HPLC at two, four, six, and eight weeks.

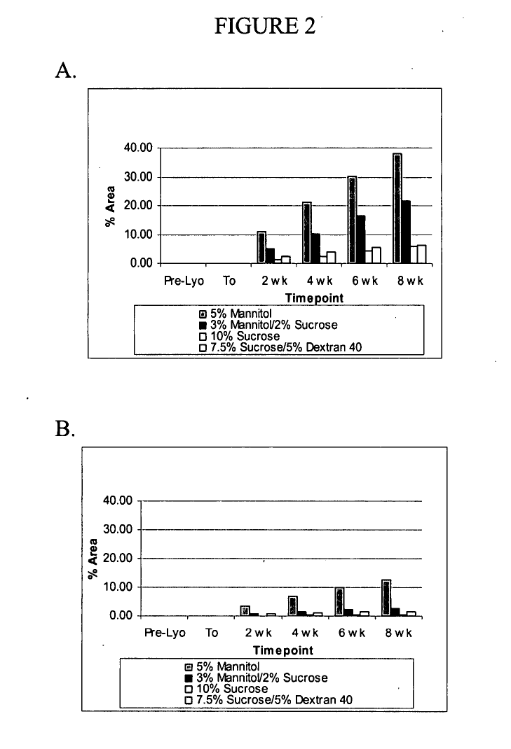

Pyroglutamate (RP-HPLC) data from the samples kept at 40° C. are depicted in FIG. 2 (panel A is PBS buffer, panel B is histidine buffer). Although mannitol is generally considered a stabilizing bulking agent/cryoprotectant, mannitol is destabilizing in ecallantide formulations, as shown in FIG. 2. Formulations containing mannitol as the sole bulking agent/cryoprotectant had considerably greater levels of pyroglutamate than the others formulations, and while stability of formulations containing a mixture of sucrose and mannitol was better than those having mannitol alone, these formulations still had greater levels of pyroglutamate than the sucrose and sucrose/dextran formulations. SEC-HPLC data for aggregate and fragmentation products was similar.

### Example 4

### Bulking Agent/Cryoprotectant Selection for Increased Dose Ecallantide

Ecallantide stability was examined in lyophilized formulations utilizing different bulking agent/cryoprotectant schemes. Ecallantide (20 mg/mL in PBS, pH 7.0) was buffer-exchanged by dialysis into formulations buffered with 10 mM histidine, pH 6.5 and including (a) 10% sucrose, (b) 3% mannitol/3% sucrose, or (c) 3% mannitol/3% trehalose as a bulking agent/cryoprotectant.

Samples of each formulation were sterile filtered into glass vials, frozen, lyophilized by freezing to −40° C. in a lyophilizer, then primary drying at 75 mTorr at −40° C. for 30 minutes, −25° C. for 23 hours, 5° C. for 10 hours, then secondary drying at 75 mTorr, 30° C. for 9 hours. The lyophilized samples were stored at 4° or 40° C. for eight weeks. Samples were assayed by SEC-HPLC and RP-HPLC at two (40° samples only), four, six, and eight weeks.

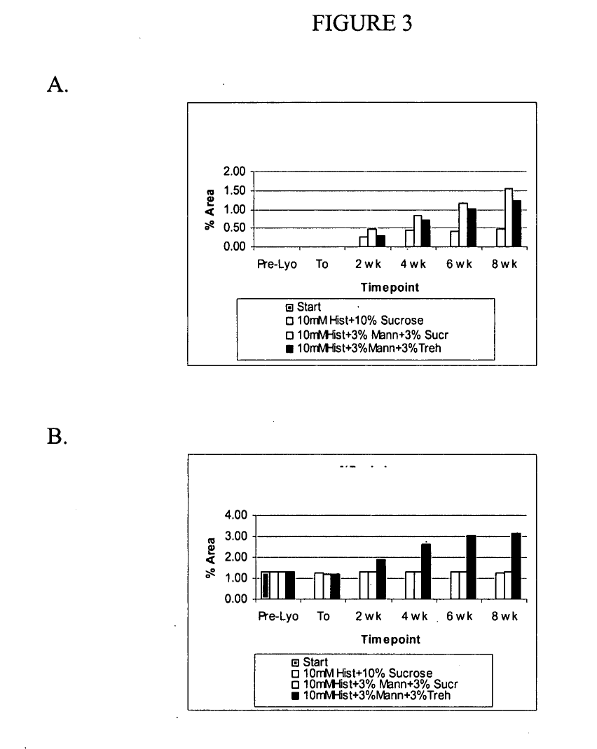

RP-HPLC and SEC-HPLC analysis showed that the samples containing 10% sucrose as the bulking agent/cryoprotectant had considerably less degradation than the sucrose/mannitol and mannitol/trehalose formulations. As shown in FIG. 3, the mannitol-containing formulations had greater amounts of pyroglutamate (panel A) and “peak 4” contaminant (panel B: “peak 4” is believed to be a mixture of oxidized and glycosylated ecallantide that cannot be resolved by this method).

It will be understood by those skilled in the art that additional substitutions, modifications and variations of the described embodiments and features may be made without departing from the invention as described above or as defined by the appended claims.

The publications cited herein are hereby incorporated by reference in their entireties.

What is claimed is:

1. An ecallantide formulation, comprising
a buffering agent selected from the group consisting of histidine and phosphate;
a bulking agent/cryoprotectant selected from the group consisting of sucrose and a combination of sucrose and mannitol; and
ecallantide,
said formulation having a pH of about 6.0 to 7.0.

2. The formulation of claim 1, wherein said buffering agent is histidine

3. The formulation of claim 2, wherein said bulking agent/cryoprotectant is sucrose.

4. The formulation of claim 3, wherein said pH is about pH 6.5.

5. The formulation of claim 4, wherein the buffering agent and the ecallantide are present at a molar ratio of 2:1 to 2.5:1 (buffering agent:ecallantide).

6. The formulation of claim 5, wherein the bulking agent/cryoprotectant and the ecallantide are present at a molar ratio of 75:1 to 60:1 (bulking agent/cryoprotectant:ecallantide).

7. The formulation of claim 6, wherein the buffering agent, the bulking agent/cryoprotectant, and the ecallantide are present at a molar ratio of 2.5:75:1 to 2:65:1 (buffering agent:bulking agent/cryoprotectant:ecallantide).

8. The formulation of claim 1, wherein said bulking agent/cryoprotectant is sucrose.

9. The formulation of claim 1, wherein said pH is about pH 6.5.

10. The formulation of claim 1, wherein the formulation is lyophilized.

11. The formulation of claim 1, wherein the buffering agent and the ecallantide are present at a molar ratio of 1:1 to 7.5:1 (buffering agent:ecallantide).

12. The formulation of claim 11, wherein the buffering agent and the ecallantide are present at a molar ratio of 2:1 to 2.5:1 (buffering agent:ecallantide).

13. The formulation of claim 1, wherein the bulking agent/cryoprotectant and the ecallantide are present at a molar ratio of 300:1 to 45:1 (bulking agent/cryoprotectant:ecallantide).

14. The formulation of claim 13, wherein the bulking agent/cryoprotectant and the ecallantide are present at a molar ratio of 75:1 to 60:1 (bulking agent/cryoprotectant:ecallantide).

15. The formulation of claim 1, wherein the buffering agent, the bulking agent/cryoprotectant, and the ecallantide are present at a molar ratio of 2.5:75:1 to 2:65:1 (buffering agent:bulking agent/cryoprotectant:ecallantide).

16. The formulation of claim 1, consisting essentially of:
a buffering agent selected from the group consisting of histidine and phosphate;
a bulking agent/cryoprotectant selected from the group consisting of sucrose and a combination of sucrose and mannitol; and
ecallantide.

17. A lyophilized ecallantide formulation, comprising:
a buffering agent selected from the group consisting of histidine and phosphate;
a bulking agent/cryoprotectant selected from the group consisting of sucrose and a combination of sucrose and mannitol; and
ecallantide.

18. The formulation of claim 17, wherein said buffering agent is histidine

19. The formulation of claim 18, wherein said bulking agent/cryoprotectant is sucrose.

20. The formulation of claim 19, wherein pH of the reconstituted formulation is about pH 6.5.

21. The formulation of claim 20, wherein the buffering agent and the ecallantide are present at a molar ratio of 2:1 to 2.5:1 (buffering agent:ecallantide).

22. The formulation of claim 21, wherein the bulking agent/cryoprotectant and the ecallantide are present at a molar ratio of 75:1 to 60:1 (bulking agent/cryoprotectant:ecallantide).

23. The formulation of claim 22, wherein the buffering agent, the bulking agent/cryoprotectant, and the ecallantide are present at a molar ratio of 2.5:75:1 to 2:65:1 (buffering agent:bulking agent/cryoprotectant:ecallantide).

24. The formulation of claim 17, wherein said bulking agent/cryoprotectant is sucrose.

25. The formulation of claim 17, wherein said pH is about pH 6.5.

26. The formulation of claim 17, wherein the formulation is lyophilized.

27. The formulation of claim 17, wherein the buffering agent and the ecallantide are present at a molar ratio of 1:1 to 7.5:1 (buffering agent:ecallantide).

28. The formulation of claim 27, wherein the buffering agent and the ecallantide are present at a molar ratio of 2:1 to 2.5:1 (buffering agent:ecallantide).

29. The formulation of claim 17, wherein the bulking agent/cryoprotectant and the ecallantide are present at a molar ratio of 300:1 to 45:1 (bulking agent/cryoprotectant:ecallantide).

30. The formulation of claim 29, wherein the bulking agent/cryoprotectant and the ecallantide are present at a molar ratio of 75:1 to 60:1 (bulking agent/cryoprotectant:ecallantide).

31. The formulation of claim 17, wherein the buffering agent, the bulking agent/cryoprotectant, and the ecallantide are present at a molar ratio of 2.5:75:1 to 2:65:1 (buffering agent:bulking agent/cryoprotectant:ecallantide).

32. A lyophilized ecallantide formulation, produced by the process of:
(a) obtaining a mixture of a buffering agent selected from the group consisting of histidine and phosphate, a bulking agent/cryoprotectant selected from the group consisting of sucrose and a combination of sucrose and mannitol; and ecallantide; and
(b) lyophilizing said mixture.

33. A method for making a lyophilized ecallantide formulation, produced by the process of:
(a) obtaining a mixture of a buffering agent selected from the group consisting of histidine and phosphate, a bulking agent/cryoprotectant selected from the group consisting of sucrose and a combination of sucrose and mannitol; and ecallantide; and
(b) lyophilizing said mixture.
